(19) United States
(12) Patent Application Publication
(10) Pub. No.: US 2022/0267775 A1
(43) Pub. Date: Aug. 25, 2022

(54) CANCER TREATMENT USING TARGETED siRNA PHARMACEUTICAL FORMULATIONS TO DOWNREGULATE EXPRESSION OF PRDM14 PROTEIN

(71) Applicant: ARIZ Precision Medicine, Inc., Davis, CA (US)

(72) Inventors: Lonnie L. BOOKBINDER, Davis, CA (US); Brad NILE, Davis, CA (US); Nicole NUNEZ, Vacaville, CA (US); Nicole HALMAI, Davis, CA (US); Narendra K. VAISH, Kirkland, WA (US)

(21) Appl. No.: 17/571,251

(22) Filed: Jan. 7, 2022

Related U.S. Application Data
(63) Continuation of application No. PCT/US2020/041473, filed on Jul. 9, 2020.
(60) Provisional application No. 62/872,084, filed on Jul. 9, 2019.

(51) Int. Cl. C12N 15/113; A61K 45/06; A61K 47/02; A61K 47/54; A61K 47/64

(52) U.S. Cl. CPC C12N 15/1137; A61K 45/06; A61K 47/02; A61K 47/549; A61K 47/64; C12N 2310/14; C12N 2310/321; C12N 2310/3513

(57) ABSTRACT

Pharmaceutical formulations for the treatment of cancer comprising novel siRNAs that downregulate expression of the PRDM oncoprotein gene and inhibit tumor growth. siRNAs designed and selected to destroy PRDM14 mRNA are described. The siRNAs are delivered via one or more targeted drug delivery systems equipped with a tumor-specific targeting ligand that confers specific binding of the nanoparticle with siRNA payload to receptors on the surface of tumor cells.

### CROSS REFERENCE TO RELATED APPLICATIONS

This application claims benefit of U.S. provisional patent application 62/872,084, filed Jul. 9, 2019.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### FIELD OF THE INVENTION

The present invention is in the field of medicine. In particular, the invention relates to cancer treatment.

### CROSS-REFERENCE TO RELATED APPLICATIONS

This application is a Continuation Application of PCT/US2020/041473, filed on Jul. 9, 2020, and published on Jan. 14, 2021 as WO2021007465, which claims the priority to the U.S. Provisional Application No. 62/872,084, filed Jul. 9, 2019; all of which applications are incorporated herein by reference in their entirety.

### SEQUENCE LISTING

The instant application contains a Sequence Listing which has been filed electronically in ASCII format and is hereby incorporated by reference in its entirety. Said ASCII copy, created on Feb. 16, 2022, is named 60673-702_301_SL.txt and is 12,445 bytes in size.

### BACKGROUND

Current anti-cancer drug therapies are of limited effectiveness. Commonly used anti-cancer drugs may bring about temporary remission of tumors and help prolong a patient's life, but in most cases are not curative. They often do not eliminate the cancer completely and tumors may subsequently reemerge. Small molecule chemotherapies are currently the most widely used treatment for cancer, but their action is largely non-specific. Chemotherapy agents have serious off-target toxic effects, causing collateral damage to normal, healthy cells and tissues. Consequently, patients suffer from severe side effects due to toxicity Immune based therapies were expected to decrease toxicity and improve survival, but despite their promise, they have only incrementally improved the prospects for cancer treatment and for favorable long-term patient outcomes. Although targeted therapy and immunotherapy are increasingly used as supplements or alternatives to traditional chemotherapy, such therapies generally lack long-term effectiveness, because cancers commonly adapt and rapidly develop resistance, escaping the targeted therapy effect. For the foregoing reasons, chemotherapy, immunotherapy, and targeted therapies are rarely curative.

The Cancer Statistics Center of the American Cancer Society estimates 1,762,450 new cancer cases and 606,880 cancer deaths for 2019. The high mortality rate due to cancer highlights the urgent need for newer therapeutic modalities that can effectively forestall cancer progression. Fifteen percent of all new cancer cases in the United States are female breast cancer (Cancer.gov). According to the National Institutes of Health, these breast cancer cases are expected to grow by more than 50% each year, and the rate of new diagnoses is predicted to exceed 440,000 women annually by 2030 (Cancer.gov). In addition, over 40,000 women with breast cancer will die as a result of this disease in 2017 alone (Cancer.gov). These staggering numbers highlight a need for new interventions to battle this complex disease. According to the American Cancer Society, most breast cancer chemotherapeutics are nonspecific and targeted chemotherapies are applicable in less than 30% of breast cancer cases (Cancer.org). Furthermore, only 5% of breast cancers arise due to the inheritance of genetic defects, such as single point mutations, warranting further investigation into the onset and treatment of breast cancers that arise due to other factors, such as epigenetics, wherein changes in organisms are caused by modification of gene expression rather than alteration of the genetic code itself

Small interfering RNA (siRNA) has the potential to specifically silence target genes in a cell. One of the main obstacles to the successful implementation of siRNA therapy is the difficulty in delivering effective amounts of siRNA to relevant sites of action in the body. siRNAs are small molecules, on the order of only 21-23 nucleotides, and are prone to elimination from the body by the kidney, degradation by endogenous enzymes such as nucleases and lysozymes, and attack by the immune system. Additionally, the therapeutic activity of naked siRNAs is difficult to harness, as they have very short in vivo half-lives and rapid body clearance. Hence, success will be driven by an ability to match appropriate siRNAs to suppress certain problematic genes while providing a delivery system that maximizes the likelihood of delivering the novel siRNA to the targeted site, e.g., the cancer cell.

In view of the above, there is a need for molecular compositions and methods for modulating and inhibiting the expression of genes involved in cancer and furthering the capability and effectiveness of siRNA-based treatment. The present invention addresses these and other needs.

### SUMMARY OF THE INVENTION

The present invention includes pharmaceutical compositions comprising siRNA molecules that target genes expressed in cancer and methods of using such compositions to silence expression of such genes, thereby inhibiting the production of cancerous cells. Embodiments of the present invention comprise new chemical entities comprising targeted drug delivery systems loaded with siRNA designed to knock down expression of the PRDM14 gene. PRDM14 is a master regulatory gene that encodes the PRDM14 protein. Dysregulation of PRDM14 is strongly implicated in several cancer types, including breast and lung cancer, as well as other cancers.

The development of new drugs, according to the present invention, including associated drug delivery systems, designed to regulate the expression of the PRDM14 gene, is expected to fulfill a pressing need for the effective long-term treatment and even cure of certain cancer types, a need that available chemotherapy and immunotherapies are generally incapable of meeting. These new drugs, according to the present invention, will offer an effective solution that can be potentiated by using complementary drug delivery systems targeted against specific cancer cells, thereby minimizing toxic side-effects and damage to healthy cells and tissues.

The present invention provides novel siRNAs in a drug formulation with a combination of features optimized, in one instance, to treat cancer types driven by PRDM14 gene dysregulation. The invention provides for the complexation of select anti-PRDM14 siRNA therapeutic payloads into one or more delivery systems arrayed with a cancer-cell specific targeting ligand.

In some embodiments, the invention comprises siRNA in concert with nanoparticle drug delivery vehicles to preferentially deliver siRNA to cancer cell sites. The nanoparticle drug delivery system protects the siRNA from degradation and elimination during circulation and mediates internalization of the siRNA into targeted cancer cells. Additionally, the drug delivery system according to the present invention may include a targeting ligand displayed on the nanoparticle surface.

In other embodiments, the drug delivery system according to the present invention uses a non-particulate carrier (for example, an aptamer) to selectively target the siRNA to specific types of cancer cells.

The targeting ligand targets a distinctive receptor or other moiety that is highly expressed on the surface of a tumor cell or cancer stem cell. The targeting ligand distinguishes the cancerous cells from normal non-cancerous cells. The ligand may be a protein, peptide, aptamer, or other class of molecule that can bind to the targeted cancer cell with preferable high specificity and affinity. The targeted approach distinguishes cancerous cells from normal healthy cells, resulting in preferential delivery and accumulation of the drug at cancer sites, thus enhancing the therapeutic effect and minimizing toxicity to the patient.

In addition to one or more anti-PRDM14 siRNAs, the drug delivery system may incorporate other therapeutic agents such as chemotherapeutics, or any companion payload that helps inhibit, kill, or stop the proliferation and spread of cancer cells.

By offering the above-described novel combination of features, the present invention fulfills an urgent need by providing an effective pharmaceutical composition for treatment of certain cancers, including those that are driven by dysregulated PRDM14. The invention addresses the shortcomings of current treatment options and offers relief to innumerable persons suffering from certain cancers.

### BRIEF DESCRIPTION OF THE DRAWING FIGURES

FIG. 1 depicts an example according to one embodiment of the invention comprising a nanoparticle drug delivery system containing an siRNA payload with complementarity to PRDM14 mRNA.

FIG. 2 depicts the siRNA delivery system comprising a cyclized peptide targeting ligand with amino acid sequence DMPGTVLPD (SEQ ID NO: 29), which binds with high specificity and affinity to breast cancer cells, according to the inventive subject matter.

FIG. 3 shows cell viability assay results for breast cancer cells (cell line MCF-7) after transfection with anti-PRDM14 siRNAs, according to the present invention.

FIG. 4 shows cell viability assay results for control cells (cell line CCD112, normal colon fibroblast cells) after transfection with anti-PRDM14 siRNAs, according to the present invention.

FIG. 5 shows cell viability assay results for lung cancer cells (cell line A549) after transfection with anti-PRDM14 siRNAs, according to the present invention.

### DETAILED DESCRIPTION OF THE INVENTION

Embodiments of the present invention comprise new chemical entities for treatment of various cancers in humans and animals In particular, the present invention comprises targeted drug delivery systems loaded with siRNA designed to silence expression of the PRDM14 gene, thereby inhibiting cancerous growth.

In a first aspect, the present invention comprises isolated oligoribonucleotides (siRNAs) that are 16-30 nucleotides in length having a sequence that is complementary to a contiguous portion of the human PRDM14 gene sequence (identified herein as SEQ ID NO: 1 and as RefSeq Accession No.: NM_024504).

Table 1, below, lists siRNA sequences designed, according to the inventive subject matter, to downregulate expression of the PRDM14 gene and inhibit cancer growth. In certain embodiments, site-specific base and backbone modifications may be designed into the siRNAs to protect the siRNA from degradation and elimination, prevent immunogenicity, and minimize the potential for off-target effects.

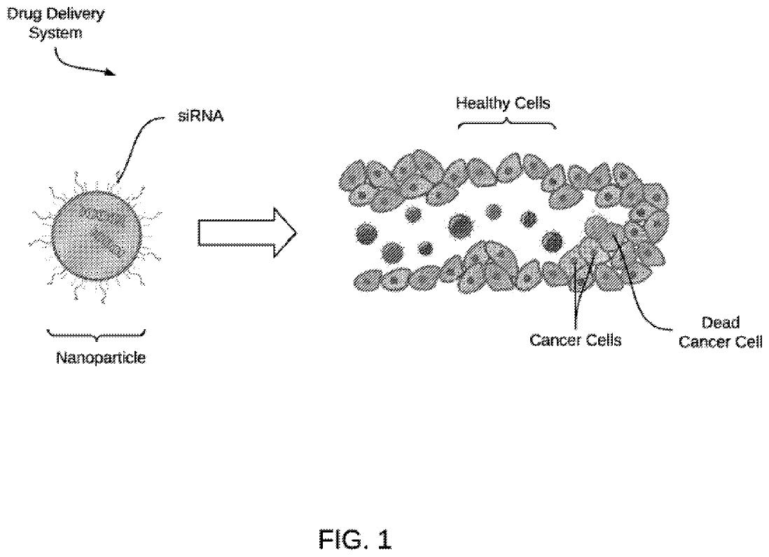

FIG. 1 depicts an example according to one embodiment of the invention comprising a nanoparticle drug delivery system containing an siRNA payload with complementarity to PRDM14 mRNA. The nanoparticle acts as a carrier and drug delivery system for the siRNA. The nanoparticle includes targeting agents that have affinity for specific types of cancer cells, rather than healthy cells. Once the nanoparticle binds with a cancer cell, the nanoparticle releases the siRNA into the cancer cell, causing its death. A plurality of nanoparticles with siRNA and targeting agents are introduced into a subject in a dose sufficient to effectively kill targeted cancer cells while avoiding harm to healthy cells.

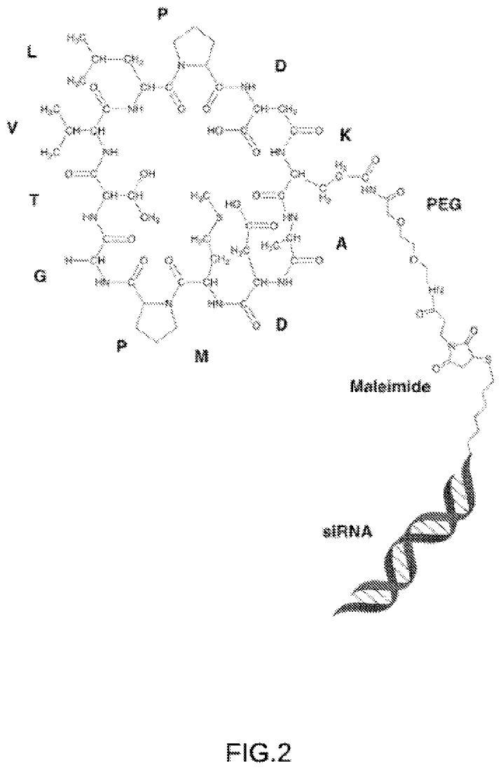

FIG. 2 depicts the siRNA delivery system according to the inventive subject matter comprising a cyclized peptide targeting ligand with amino acid sequence DMPGTVLPD (SEQ ID NO: 29), which binds with high specificity and affinity to breast cancer cells. This anti-PRDM14 siRNA ARIZ-026 (SEQ ID NOs: 10 and 11), which is discussed further below in relation to a fifth embodiment, may be conjugated to the cyclized targeting ligand c(DMPGTVLPD) (SEQ ID NO: 29) via a thiol-maleimide-PEG linker. c(DMPGTVLPD) (SEQ ID NO: 29) is a cyclized version of the DMPGTVLPD peptide (SEQ ID NO: 29) demonstrated to bind to breast cancer cells with high specificity and affinity. The resultant c(DMPGTVLPD)-PEG-MAL-siRNA molecules (“DMPGTVLPD” disclosed as SEQ ID NO: 29) are illustrated in FIG. 2.

In certain embodiments, the pharmaceutical compositions of the invention may have a single siRNA payload. In other embodiments, the pharmaceutical composition may comprise more than one siRNA payload to enhance therapeutic efficacy. In yet other embodiments, the pharmaceutical composition may further comprise additional siRNAs directed against target genes other than PRDM14 to enhance cancer treatment efficacy. In still other embodiments, the pharmaceutical composition may further comprise conventional anticancer agents such as chemotherapeutic agents, or any other companion payload that may enhance treatment efficacy. The inventive subject matter also includes various means for introduction of the pharmaceutical composition to a subject including intra-tumoral injection, subcutaneous injection, intravenously and via inhalation. The selected method for patient treatment will be determined based on the patient's status and type of cancer.

The siRNAs listed in Table 1 below, when delivered to a patient, may be enhanced via a drug delivery system such as a liposome, or a nanoparticle composed of lipids, cyclodextrin, chitosan, carbohydrate polymers, elastin-like polymers (ELP), calcium phosphate polymers, or combinations thereof.

The siRNA, along with any companion payloads, are delivered preferentially to cancer cells via a cancer-specific targeting nanoparticle or other type of cancer-targeting drug delivery system such as an aptamer to avoid damaging normal, healthy cells and to protect the naked siRNA from degradation, stabilize the entire payload in the circulatory system, and mediate the efficient internalization of the siRNA into cancer cells.

The nanoparticles associated with delivery of the siRNA may be assembled from a combination of carrier materials that may include, but are not limited to lipids, liposomes, sugars, dextran, calcium phosphate, chitosan, peptides, and plastic polymers.

In certain embodiments, the pharmaceutical composition further includes a cancer cell targeting ligand to enhance selectivity of the siRNA payload for cancer cells over normal healthy cells. The targeting ligand is selected to bind specifically to a receptor or other moiety that is overexpressed on the cancer cell surface and distinguishes cancer cells from normal cells. In certain embodiments, the targeting ligand may be, but is not limited to being, a polypeptide, an aptamer, or other class of molecule that could bind to the targeted cancer cell with high specificity and affinity.

In certain embodiments, the targeting ligand may be, but is not limited to being, a peptide (for example, the peptide DMPGTVLP (SEQ ID NO: 30), in a linear or cyclic form, to bind with high specificity and affinity to breast cancer cells).

In certain embodiments, a nano-pharmaceutical particle is formed by complexation of the siRNA with a calcium phosphosilicate carrier material. In other embodiments, the nanoparticle may be assembled from an Elastin-Like Polypeptide (ELP). In still other embodiments, the drug delivery system may be liposomal. In some embodiments, the drug delivery system may be coated with a protective layer (for example, PEGylation of liposomes) to extend the drug lifetime in the bloodstream and shield the drug delivery system from destruction by the immune system.

The inventive subject matter is primarily directed towards treating cancers driven by dysregulation of the PRDM14 gene, however other cancers may be impacted. In certain embodiments, the pharmaceutical composition may be formulated for, but is not limited to, treating patients suffering from breast, lung, esophagus, pancreas, ovary, kidney, bladder, renal, germ cell, leukemia, head and neck, or cervical cancer.

In a preferred embodiment, an anti-PRDM14 siRNA with site-specific base and backbone modifications is complexed into an ELP-based nanoparticle along with a chemotherapeutic companion payload and targeted to breast cancer cells using a DMPGTVLP targeting ligand (SEQ ID NO: 30).

Table 1 provided below is a listing of those siRNA duplexes directed against PRDM14 expression according to the inventive subject matter, where 4=2′OMe-U; bold=phosphorothioate; and, P=phosphate.

TABLE 1

| siRNA | | | |
|---|---|---|---|
| IDENTIFIER | SEQUENCE | STRAND | SEQ ID NO |
| ARIZ-022 | 5′-GGACAAGGGCGAUAGGAAAdTdT-3′ | Sense | 2 |
| | 3′-dTdTCCUGUUCCCGCUAUCCUUU-5′ | Antisense | 3 |
| ARIZ-023 | 5′-GCAAAGAGAUCCAUCAGAAUU-3′ | Sense | 4 |
| | 3′-UUCGUUUCUCUAGGUAGUCUU-5′ | Antisense | 5 |
| ARIZ-024 | 5′-CCAGUGAAGUGAAGACCUAdTdT-3′ | Sense | 6 |
| | 3′-dTdTGGUCACUUCACUUCUGGAU-5′ | Antisense | 7 |
| ARIZ-025 | 5′-GAGAUAAGCACCUCAAGUAdTdT-3′ | Sense | 8 |
| | 3′-dTdTCUCUAUUCGUGGAGUUCAU-5′ | Antisense | 9 |
| ARIZ-026 | 5′-AGACCUACGGAGACAAUUCUGUdTdT-3′ | Sense | 10 |
| | 3′-dTdTUUCUGGAUGCCUCUGUUAAGACA-5′ | Antisense | 11 |
| ARIZ-032 | 5′-GUGAAGACCUACGGAGACAdTdT-3′ | Sense | 12 |
| | 3′-dTdTCACUUCUGGAUGCCUCUGU-5′ | Antisense | 13 |
| ARIZ-033 | 5′-UUCUGUGAUGUGGGAGAUCdTdT-3′ | Sense | 14 |
| | 3′-dTdTAAGACACUACACCCUCUAG-5′ | Antisense | 15 |
| ARIZ-034 | 5′-UUUCCCUGUUCUCUCUGCAdTdT-3′ | Sense | 16 |
| | 3′-dTdTAAAGGGACAAGAGAGACGU-5′ | Antisense | 17 |
| ARIZ-038 | 5′-GGUAUUUACCUACAAAUAUUU-3′ | Sense | 18 |
| | 3′-UUCCAUAAAUGGAUGUUUAUA-P-5′ | Antisense | 19 |
| ARIZ-039 | 5′-GAUGGUCAUUUGAGCCACUdTdT-3′ | Sense | 20 |
| | 3′-dTdTCUACCAGUAAACUCGGUGA-P-5′ | Antisense | 21 |
| ARIZ-040 | 5′-UGGUCAUUUGAGCCACUUUAUdTdT-3′ | Sense | 22 |
| | 3′-dTdTACCAGUAAACUCGGUGAAAUA-P-5′ | Antisense | 23 |
| ARIZ-044 | 5′-P-GUGAAGACUACGGAGACAdTdT-3′ | Sense | 24 |
| | 3′-dTdTCACUUCUGGAUGCCUCUGU-5′ | Antisense | 25 |
| ARIZ-061 | 5′-GUGAAGACC4ACGGAGACAdTdT-3′ | Sense | 26 |
| | 3′-dTdTCACUUCUGGAUGCC4C4G4-P-5′ | Antisense | 27 |

The following embodiments describe the anticipated use and efficacy of the pharmaceutical compositions according to the inventive subject matter.

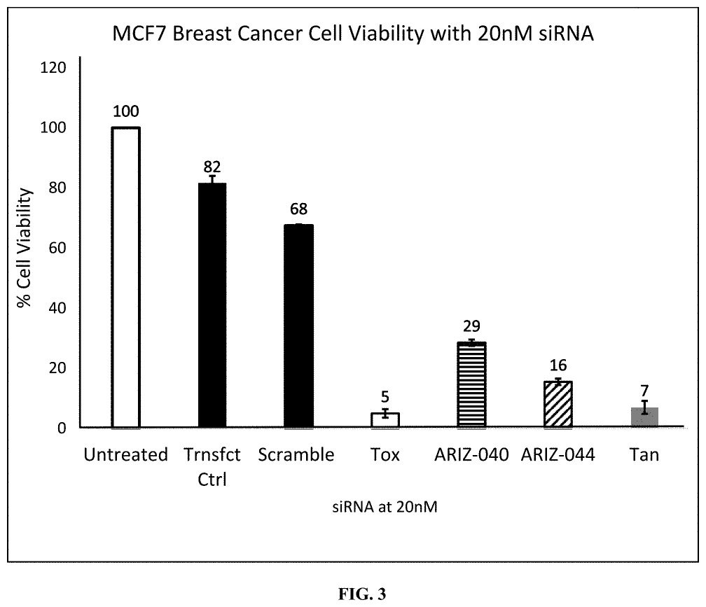

In a first embodiment according to the inventive subject matter, a composition is described illustrating the use and efficacy of siRNAs according to the present invention to kill human breast cancer cells (cell line MCF-7). Results shown in the chart of FIG. 3 reflect in vitro experimentation.

MCF-7 cancer cells were plated in a 96-well plate (1,000 cells/well) and after 24 hours, the MCF-7 cancer cells were transfected with 20 nM siRNA (including ARIZ-040 (SEQ ID NOs: 22 and 23), ARIZ-044 (SEQ ID NOs: 24 and 25), and ARIZ-026 (SEQ ID NOs: 10 and 11), using 0.3 μl Lipofectamine (Invitrogen, Inc.). Media was changed after two days, and after four days, percent cell viability was determined, relative to untreated cells. As a control, normal colon fibroblast cells (cell line CC112) were treated with the same set of siRNAs.

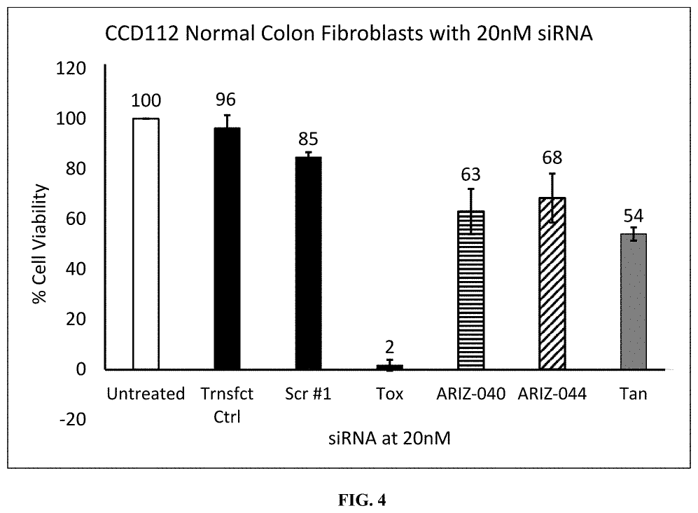

Referring now to the charts of FIG. 3 and FIG. 4, we illustrate the resulting cell viability of MCF-7 breast cancer cells compared to normal colon fibroblast cells (cell line CCD112) following exposure to our various siRNAs, including ARIZ-040 (SEQ ID NOs: 22 and 23), ARIZ-0044 (SEQ ID NOs: 24 and 25), and ARIZ-026 (SEQ ID NOs: 10 and 11).

Thus, as illustrated, anti-PRDM14 siRNAs (ARIZ-040 (SEQ ID NOs: 22 and 23), ARIZ-0044 (SEQ ID NOs: 24 and 25), and ARIZ-026 (SEQ ID NOs: 10 and 11)) were effective in killing MCF-7 breast cancer cells relative to a scrambled siRNA negative control. The results further demonstrate that the anti-PRDM14 siRNAs according to the inventive subject matter and this first embodiment specifically attack cancer cells, as killing of normal colon fibroblast cells was minimal

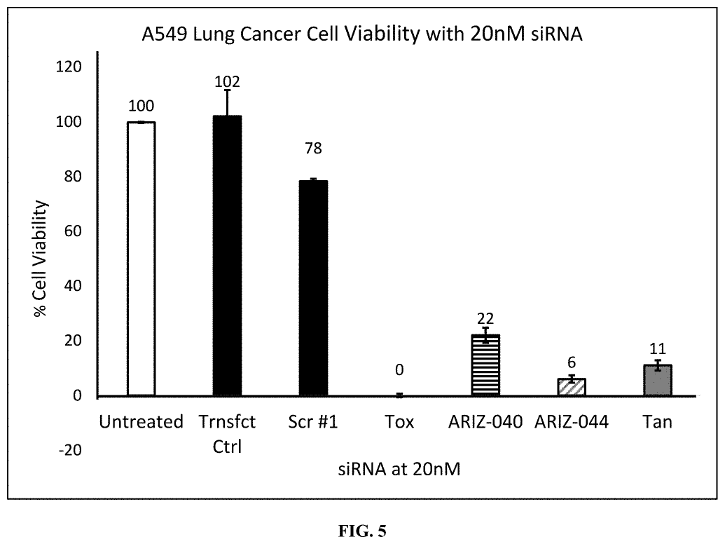

Referring now to FIG. 5, in a second embodiment according to the inventive subject matter, a composition is described illustrating the use and efficacy of siRNAs to kill human lung cancer cells (cell A549). Results shown in the chart of FIG. 4 reflect in vitro results.

A549 cancer cells were plated in a 96 well plate (1,000 cells/well) and after 24 hours, the A549 cancer cells were transfected with 20 nM siRNAs (ARIZ-040 (SEQ ID NOs: 22 and 23), ARIZ-0044 (SEQ ID NOs: 24 and 25), and ARIZ-026 (SEQ ID NOs: 10 and 11)), using 0.3 μl Lipofectamine (Invitrogen, Inc.). Media was changed after two days, and after four days, percent cell viability was determined, relative to untreated cells.

The chart of FIG. 5 provides an illustration of the resulting cell viability of A549 lung cancer cells following exposure to the siRNAs, including ARIZ-040 (SEQ ID NOs: 22 and 23), ARIZ-0044 (SEQ ID NOs: 24 and 25), and ARIZ-026 (SEQ ID NOs: 10 and 11). Thus, Anti-PRDM14 siRNAs according to the present invention, were effective in killing A549 lung cancer cells relative to a scrambled siRNA negative control.

In a third embodiment, the described inventive subject matter is particularly directed to breast cancer cells. To illustrate a potential clinical analog for administration of a pharmaceutical composition according to a third embodiment of the present invention, wherein an anti-PRDM14 siRNA (ARIZ-061 (SEQ ID NOs: 26 and 27)) with site-specific base and backbone modifications may be complexed into an ELP-based nanoparticle along with a chemotherapeutic companion payload bound to the targeted nanoparticle via a drug-binding domain, and subsequently targeted to breast cancer cells with a DMPGTVLP targeting ligand (SEQ ID NO: 30).

The third embodiment comprises a protein nanoparticle system for targeting siRNA or other drugs into tumors. The basis of the delivery system is elastin-like peptides (ELP) that self-assemble once exposed to the nucleic acid of the siRNA. Specific targeting peptides are fused to the core ELP structure by standard genetic engineering techniques. The ELP comprises a cationic nucleic acid binding domain (NBD) for binding of the negatively charged siRNA; an assembly domain (AD) that governs the self-assembly of individual polypeptide molecules into a nanoparticle; and a cell targeting domain (CTD) that comprises the peptide targeting ligand. The ELP may also comprise a drug binding domain (DBD) to allow for complexing a companion payload (for example, a chemotherapeutic agent) for greater therapeutic effect.

ELP-based nanoparticles may be formed using an ELP construct engineered to contain the targeting peptide DMPGTVLP (SEQ ID NO: 30) within the CTD, and a drug binding domain that binds the chemotherapy drug doxorubicin. The DMPGTVLP ligand (SEQ ID NO: 30) will target breast cancer cells by binding to breast cancer cells with high specificity and affinity. The nanoparticles are formed in a complex with the anti-PRDM14 siRNA ARIZ-061 (SEQ ID NOs: 26 and 27) and a therapeutically effective amount of doxorubicin. To assess the designed siRNA potency, the drug formulation thus produced is administered to nude mice with tumors formed by subcutaneously injecting female mice with three million breast metastatic tumor cells and permitting tumors to grow over a 10-day period. The treatment consists of up to five injections of the drug formulation, each injection delivering 10 microliters of 1 nmol siRNA immediately into the tumors or 5 mg/kg siRNA intravenously. An equal number of mice bearing similar tumors are injected with nanoparticles containing a scrambled siRNA sequence as a negative control. Treated tumors are then removed after four weeks of dosing, measured, and weighed. Expression of PRDM14 mRNA in the tumors would be measured by qPCR using standard analytical methods. Expression of PRDM14 protein in the tumors would be measured by Western blotting using standard analytical methods.

Tumors from mice dosed with the novel formulation containing anti-PRDM siRNA, ARIZ-061, according to the inventive subject matter will be 50% to 90% smaller in size and weight than tumors from mice treated with the scrambled siRNA. Expression of PRDM14 mRNA and PRDM14 protein in tumors from mice treated with the anti-PRDM siRNA formulation based on ARIZ-061 will be reduced by 50% to 90% compared to the negative controls.

In a fourth embodiment, the described inventive subject matter is particularly directed to lung cancer cells. This fourth embodiment comprises a targeted, self-assembled nanoparticle drug delivery system for cancer treatment leveraging siRNA according to the inventive subject matter (ARIZ-044 (SEQ ID NOs: 24 and 25)), wherein the siRNA is directly conjugated to a cyclic peptide targeting ligand via a thiol-maleimide-PEG linker, and the nanoparticle is capable of delivering the siRNA payload specifically to cancer cells expressing the target receptor.

This fourth embodiment leverages a small cyclopeptide (cyclo(Arg-Gly-Asp)-d-Phe- Lys[PEG-MAL]) (cRGD) as a ligand to specifically target siRNA-bearing nanoparticles to human lung cancer tumors. cRGD specifically targets the integrin αvβ33 receptor, which in humans is overexpressed on many solid tumor types, including lung cancer tumors. c(RGD) is conjugated to siRNA directed against the VEGF receptor 2 (VEGFR2). VEGFR2 is involved in signaling pathways that result in the proliferation and migration of endothelial vessels, and consequently promote angiogenesis and vascular growth of tumors. The c(RGD)/siRNA nanoparticles carry the siRNA into A549 lung cancer cells and silence the VEGFR2 gene both in vitro and in vivo. In tumor-bearing mice, intravenously injected cRGD-siRNA molecules generate no innate immune response and bio-distribute to tumor tissues. Intravenous injection of siRNA-bearing nanoparticles inhibits tumor growth and angiogenesis in a mouse model system. Continuous systemic delivery of cRGD-siRNAs has resulted in downregulation of corresponding mRNA (45% to 50%) and protein (45% to 65%) in tumors, as well as in overall reduction of tumor volume (70% to 90%).

c(RGD)-siRNA nanoparticles are formed using the anti-PRDM14 siRNA ARIZ-044 (SEQ ID NOs: 24 and 25). The c(RGD)-siRNA is injected intravenously into mice bearing A549 lung cancer tumors. The mice are then subsequently monitored for any toxic effects of the treatment, and tumor size is measured to determine therapeutic efficacy. Expression of PRDM14 mRNA and PRDM14 protein in the tumors is analyzed.

Based on this fourth embodiment, tumors from mice treated with c(RGD)-siRNA nanoparticles bearing the anti-PRDM14 siRNA (ARIZ-044 (SEQ ID NOs: 24 and 25)) will be 70% to 90% smaller in size than tumors from control mice injected with cRGD conjugated to a control non-targeting siRNA molecule. Expression of PRDM14 mRNA and PRDM14 protein in A549 lung cancer tumors from mice treated with the c(RGD)-navigated anti-PRDM14 siRNA will be reduced by 50% or more compared to the negative controls.

In a fifth embodiment, the described inventive subject matter is particularly directed to breast cancer cells. Referring once again to FIG. 2, this fifth embodiment comprises a targeted drug delivery system for cancer treatment comprising a calcium phosphosilicate nanocarrier (NanoJacket) to display a targeting ligand to deliver an anti-PRDM14 siRNA payload (ARIZ-026 (SEQ ID NOs: 10 and 11)) specifically to cancer cells.

The calcium phosphate nanoparticles function as carrier vehicles to deliver siRNAs to targeted cancer cells for therapeutic purposes. These nanoparticles, referred to herein as “siRNA NanoJackets,” are stable and non-toxic. siRNA NanoJackets have been shown to be effective against human breast cancer cells in an animal model. Therapeutic efficiency can be increased by attaching tumor-specific targeting moieties, such as peptides, antibodies, or aptamers, to the NanoJacket surface.

The anti-PRDM14 siRNA ARIZ-026 (SEQ ID NOs: 10 and 11) may be conjugated to the cyclized targeting ligand c(DMPGTVLPD) (SEQ ID NO: 29) via a thiol-maleimide-PEG linker. c(DMPGTVLPD) (SEQ ID NO: 29) is a cyclized version of the DMPGTVLPD peptide (SEQ ID NO: 29) demonstrated to bind to breast cancer cells with high specificity and affinity. The resultant c(DMPGTVLPD)-PEG-MAL-siRNA molecules (“DMPGTVLPD” disclosed as SEQ ID NO: 29) are illustrated in FIG. 2. The c(DMPGTVLPD)-PEG-MAL-siRNA molecules (“DMPGTVLPD” disclosed as SEQ ID NO: 29) are assembled into a targeted calcium phosphate NanoJacket particle. To assess siRNA potency, the drug formulation thus produced is administered to nude mice with tumors formed by subcutaneously injecting female mice with three million breast metastatic tumor cells and permitting tumors to grow over a 10-day period. The treatment consists of up to five injections of the drug formulation, each injection delivering 10 microliters of 1 nmol siRNA into the tumors or 5 mg/kg intravenously. An equal number of mice bearing similar tumors are injected with nanoparticles containing a scrambled siRNA sequence as a negative control. Tumors are removed after four weeks of dosing, measured, and weighed. Expression of PRDM14 mRNA in the tumors is measured by qPCR using standard analytical methods. Expression of PRDM14 protein in the tumors is measured by Western blotting using standard analytical methods.

Tumors from mice dosed with the formulation containing anti-PRDM siRNA according to this fifth embodiment will be 50% to 90% smaller in size and weight than tumors from mice treated with the scrambled siRNA. Expression of PRDM14 mRNA and PRDM14 protein in tumors from mice treated with the anti-PRDM siRNA formulation will be reduced by 50% to 90% compared to the negative controls.

In a sixth embodiment, the described inventive subject matter is particularly directed to breast cancer cells. This sixth embodiment provides an illustration of the use of a targeted drug delivery system for breast cancer treatment according to the present invention, comprising an aptamer/siRNA formulation wherein the aptamer targets the nucleolin receptor and the siRNA contains a cancer-toxic timer seed sequence.

Nucleolin is a receptor known to be overexpressed in cancer cells and tumor-associated blood vessels. It has been implicated in various processes supporting tumorigenesis and angiogenesis. Its overexpression has been demonstrated in a variety of human cancers including breast cancer. An anti-PRDM14 siRNA is targeted to breast cancer cells using an RNA aptamer selected to bind strongly and specifically to the nucleolin receptor.

An RNA aptamer specifically targeting nucleolin is isolated from an RNA library using the SELEX method. The aptamer thus identified is synthesized to include a 16 nucleotide “sticky” 3′ end and coupled to an anti-PRDM14 siRNA with a complementary “sticky” end on the 3′ end of the antisense (guide) strand. The siRNA is further designed with a cancer-toxic 6 mer seed sequence.

To assess siRNA potency, the aptamer/siRNA complex thus produced is administered to nude mice with tumors formed by subcutaneously injecting female mice with three million breast metastatic tumor cells and permitting tumors to grow over a 10-day period. The treatment consists of up to five injections of the drug formulation, each injection delivering 10 microliters of 1 nmol siRNA into the tumors, or 5 mg/kg intravenously. An equal number of mice bearing similar tumors are injected with aptamer/siRNA complexes containing a scrambled siRNA sequence as a negative control. Tumors are removed after three weeks of dosing, measured, and weighed. Expression of PRDM14 mRNA in the tumors is measured by qPCR using standard analytical methods. Expression of PRDM14 protein in the tumors is measured by Western blotting using standard analytical methods.

Tumors from mice dosed with the formulation according to this sixth embodiment containing anti-PRDM siRNA according to the inventive subject matter will be 50% to 90% smaller in size and weight than tumors from mice treated with the scrambled siRNA. Expression of PRDM14 mRNA and PRDM14 protein in tumors from mice treated with the anti-PRDM siRNA formulation will be reduced by 50% to 90% compared to the negative controls.

In a seventh embodiment, the described inventive subject matter is particularly directed to breast cancer cells. This seventh embodiment illustrates a targeted drug delivery system for cancer treatment, comprising liposomal siRNA/phage fusion protein carrier complexed with anti-PRDM14 siRNA and a companion chemotherapeutic payload, wherein the complex displays a targeting ligand to deliver the siRNA payload specifically to cancer cells.

A targeted drug delivery nanocarrier comprises landscape phage fusion proteins bearing a peptide targeting ligand which self-assembles with phospholipid molecules into a liposomal particle. The nanoparticles enclose one or more drug payloads such as chemotherapeutic agents or therapeutically active polynucleotides. The phage fusion proteins are phage pVIII coat proteins displaying heterologous targeting peptides. Landscape phage fusion proteins displaying the peptide VEEGGYIAA (SEQ ID NO: 31) bind selectively to human MCF-7 breast cancer cells. These nanoparticles are leveraged to target anti-PRDM14 siRNA to MCF-7 breast cancer cells and silence PRDM14.

Liposome-based siRNA/phage fusion protein targeted particles are constructed to display the VEEGGYIAA (SEQ ID NO: 31) peptide and to deliver anti-PRDM14 siRNA to MCF-7 breast cancer cells. The nanoparticles are formulated to contain a therapeutically effective dose of the chemotherapy drug doxorubicin, along with the primary siRNA payload. The formulation thus produced is administered intravenously to mice bearing MCF-7 breast cancer tumors. To assess siRNA potency, the drug formulation thus produced is administered to nude mice with tumors formed by subcutaneously injecting female mice with three million breast metastatic tumor cells and permitting tumors to grow over a 10-day period. The treatment consists of up to five injections of the drug formulation, each injection delivering 10 microliters of 1 nmol siRNA into the tumors, or 5 mg/kg intravenously. An equal number of mice bearing similar tumors are injected with nanoparticles containing a scrambled siRNA sequence as a negative control. Tumors are removed after four weeks of dosing, measured, and weighed. Expression of PRDM14 mRNA in the tumors is measured by qPCR using standard analytical methods. Expression of PRDM14 protein in the tumors is measured by Western blotting using standard analytical methods.

Tumors from mice dosed with formulation containing anti-PRDM siRNA from Table I will be 50% to 90% smaller in size and weight than tumors from mice treated with the scrambled siRNA. Expression of PRDM14 mRNA and PRDM14 protein in tumors from mice treated with the anti-PRDM siRNA formulation will be reduced by 50% to 90% compared to the negative controls.

Although several embodiments of the inventive subject matter have been described in the foregoing detailed description, it will be understood that the invention is not limited to the embodiment(s) disclosed, but is capable of numerous rearrangements, modifications and substitutions without departing from the scope of the invention.

Throughout this specification the word “comprise”, or variations such as “comprises” or “comprising” will be understood to imply the inclusion of a stated element, integer or step, or group of elements, integers or steps, but not the exclusion of any other element, integer or step, or group of elements, integers or steps.

The associated in vitro experimentation supports assessment of the results for the described embodiments.

While the foregoing written description of the invention enables one of ordinary skill to make and use various embodiments thereof, those of ordinary skill will understand and appreciate the existence of variations, combinations, and equivalents of the specific embodiment, method, and examples herein. The invention should therefore not be limited by the above described embodiments, method, and examples, but by all embodiments and methods within the scope and spirit of the invention as claimed.

What is claimed is:

1. An isolated double-stranded small interfering RNA (siRNA) configured to inhibit the expression of the human PRDM14 gene in cancer cells, comprising SEQ ID Nos. 2 through 27, wherein the nucleotides may be RNA, DNA and a hybrid RNA:DNA combination and wherein the strands are 16-30 nucleotides in length.

2. The siRNA of claim 1 wherein the siRNA includes one or more chemical modifications comprising site-specific base modifications.

3. The siRNA of claim 2 wherein the chemical modifications comprise at least one of the following:
a. addition of an O-Methyl group to the 2′ position on the sugar;
b. addition of a fluorine atom to the 2′ position of the sugar; and
c. substitution of a sulfur atom for oxygen at the 3′ end of the siRNA guide antisense strand.

4. A pharmaceutical composition for inhibiting the expression of the PRDM14 gene in cancer cells, the pharmaceutical composition comprising:
a. a double-stranded siRNA that downregulates PRDM14 gene expression;
b. a carrier that complexes the siRNA;
c. an additional therapeutic agent; and
d. a cancer-cell specific targeting ligand.

5. The pharmaceutical composition of claim 4, wherein the carrier is calcium-phosphate based.

6. The pharmaceutical composition of claim 4, wherein the targeting ligand is selected from the group of a protein, a peptide, and an aptamer.

7. The pharmaceutical composition of claim 4, wherein the targeting ligand comprises a cyclized polypeptide.

8. The pharmaceutical composition of claim 4, wherein the targeting ligand comprises the amino acid sequence DMPGTVLP (SEQ ID NO: 30).

9. The pharmaceutical composition of claim 4, wherein the targeting ligand comprises a cyclized polypeptide comprising the amino acid sequence DMPGTVLP (SEQ ID NO: 30).

10. The pharmaceutical composition of claim 4, wherein the carrier is liposomal.

11. The pharmaceutical composition of claim 4, wherein the liposome is PEGylated.

12. The pharmaceutical composition of claim 4, further comprising an additional therapeutic agent.

13. The pharmaceutical composition of claim 4, wherein the carrier is a nanoparticle carrier comprising a polypeptide with an Elastin-Like Protein (ELP) Assembly Domain (AD), a tumor-specific Cell Targeting Domain (CTD), and a cationic Nucleic Acid Binding Domain (NBD).

14. The pharmaceutical composition of claim 11, wherein the CTD comprises the amino acid sequence DMPGTVLP (SEQ ID NO: 30).

15. The pharmaceutical composition of claims 11 further comprising a Drug Binding Domain (DBD) with a bound therapeutic agent.

16. A method for treating a human cancer patient by administering a therapeutically effective amount of a pharmaceutical composition for inhibiting the expression of the human PRDM14 gene in cancer cells, the pharmaceutical composition comprising:
a. a double-stranded siRNA that downregulates PRDM14 gene expression;
b. a carrier that complexes the siRNA and an additional therapeutic agent; and, c. a cancer cell specific targeting ligand, d. wherein the cancer cell type is selected from the group of breast cancer, lung cancer, esophageal cancer, pancreatic cancer, ovarian cancer, kidney cancer, bladder cancer, renal cancer, germ cell cancer, blood cancers, leukemia, head cancer, neck cancer and cervical cancer.

17. A method according to claim 16 wherein the carrier is calcium phosphate based.

18. A method according to claim 16 wherein the carrier is a nanoparticle carrier comprising:
a. a polypeptide with an Elastin-Like Protein (ELP);
b. an Assembly Domain (AD);
c. a tumor-specific Cell Targeting Domain (CTD); and,
d. a cationic Nucleic Acid Binding Domain (NBD).

19. A method according to claim 16 wherein the nanoparticle carrier is liposomal.

20. A pharmaceutical composition comprising a drug delivery system loaded with an ARIZ siRNA to inhibit expression of PRDM14 to suppress growth of cancer cells.

21. The pharmaceutical composition of claim 20 wherein said ARIZ siRNA is selected from the group, comprising:
a. ARIZ-022;
b. ARIZ-023;
c. ARIZ-024;
d. ARIZ-025;
e. ARIZ-026;
f. ARIZ-032;
g. ARIZ-033;
h. ARIZ-034;
i. ARIZ-038;
j. ARIZ-039;
k. ARIZ-040;
l. ARIZ-044; and,
m. ARIZ-061.


SEQUENCE LISTING (31 sequences)

SEQ ID NO: 1 - DNA, 2269 bp, Homo sapiens
gcccgaagtctaccgagcccgagtggcctaccgagcccgagtggccccgcagcgtccaggaggcgcccgctccgcggtggcgctcttggaggtggtgtcggagagccgccgagcgtgcggtcccgggatggctctaccccggccaagtgaggccgtgcctcaggacaaggtgtgctacccgccggagagcagcccgcagaacctggccgcgtactacacgcctttcccgtcctatggacactacagaaacagcctggccaccgtggaggaagacttccaacctttccggcagctggaggccgcagcgtctgctgcccccgccatgccccccttccccttccggatggcgcctcccttgctgagcccgggtctgggcctacagagggagcctctctacgatctgccctggtacagcaagctgccaccgtggtacccaattccccacgtccccagggaagtgccgcccttcctgagcagcagccacgagtacgcgggtgccagcagtgaagatctgggccaccaaatcattggtggcgacaacgagagtggcccgtgttgtggacctgacactttaattccaccgccccctgcggatgcttctctgttacctgaggggctgaggacctcccagttattaccttgctcacccagcaagcagtcagaggatggtcccaaaccctccaaccaagaagggaagtcccctgctcggttccagttcacggaggaggacctgcacttcgttctgtacggggtcactcccagcctggagcacccagccagcctgcaccatgcgatttcaggcctcctggtccccccagacagctctggatctgattctcttcctcaaactctggataaagactcccttcaacttccagaaggtctatgcctcatgcagacggtgtttggtgaagtcccacattttggtgtgttctgcagtagttttatcgccaaaggagtcaggtttgggccctttcaaggtaaagtggtcaatgccagtgaagtgaagacctacggagacaattctgtgatgtgggagatctttgaagatggtcatttgagccactttatagatggaaaaggaggtacggggaactggatgtcctatgtcaactgtgcccgcttccccaaggagcagaacctagttgctgtgcagtgtcaagggcatatattttatgagagctgcaaagagatccatcagaaccaagagctccttgtgtggtatggagactgctatgagaaatttctggatattcctgtgagccttcaggtcacagagccggggaagcagccatctgggccctctgaagagtctgcagaaggctacagatgtgaaagatgtgggaaggtatttacctacaaatattacagagataagcacctcaagtacaccccctgtgtggacaagggcgataggaaatttccctgttctctctgcaaacgatcctttgagaagcgggaccggcttcggatccacattcttcatgttcatgagaagcaccggcctcacaagtgttctacatgtgggaaatgtttctctcaatcttccagcctaaacaaacacatgcgagtccactctggagacagaccataccagtgtgtgtattgtactaagaggttcacagcctccagcatactccgcacacacatcaggcagcactccggggagaagcccttcaaatgcaagtactgtggtaaatcttttgcatcccatgctgcccatgacagccatgtccggcgttcacacaaggaggatgatggctgctcatgcagcatctgtgggaaaatcttctcagatcaagaaacattctactcccacatgaagtttcatgaagactactagccctgccaggcacaatgactcacgcctgtaatcccagcactttgggaggcagaggtgggtggatcactcaagtccaggagttcgagaccagcctgggcaacatggtgaaatcctgtctctaccaaaaaaatacaaaaatcagctgggggtggtggcacatgcctgtggttccagccactcaggaggtcgaggtggcaggatggtttgagcacaggagacggaggttgctgtgagctgagatcgccccactgcttttcaacctgggtgacagaaccagaccctgtctcaaaacaaaacaaaacaaaaaaaatgagtagccctcaagagtgtggagacaatgtaaaaacaagagattcggattctctctatttccttttatgggttatagaagtccctgcagttggctgtgtgtggtggctcacgcct

SEQ ID NO: 2 - DNA, 21 bp, Artificial Sequence
  Description of Artificial Sequence Synthetic oligonucleotide
  Description of Combined DNA/RNA Molecule Synthetic oligonucleotide
ggacaagggcgauaggaaatt

SEQ ID NO: 3 - DNA, 21 bp, Artificial Sequence
  Description of Artificial Sequence Synthetic oligonucleotide
  Description of Combined DNA/RNA Molecule Synthetic oligonucleotide
uuuccuaucgcccuugucctt

SEQ ID NO: 4 - RNA, 21 nt, Artificial Sequence
  Description of Artificial Sequence Synthetic oligonucleotide
gcaaagagauccaucagaauu

SEQ ID NO: 5 - RNA, 21 nt, Artificial Sequence
  Description of Artificial Sequence Synthetic oligonucleotide
uucugauggaucucuuugcuu

SEQ ID NO: 6 - DNA, 21 bp, Artificial Sequence
  Description of Artificial Sequence Synthetic oligonucleotide
  Description of Combined DNA/RNA Molecule Synthetic oligonucleotide
ccagugaagugaagaccuatt

SEQ ID NO: 7 - DNA, 21 bp, Artificial Sequence
  Description of Artificial Sequence Synthetic oligonucleotide
  Description of Combined DNA/RNA Molecule Synthetic oligonucleotide
uaggucuucacuucacuggtt

SEQ ID NO: 8 - DNA, 21 bp, Artificial Sequence
  Description of Artificial Sequence Synthetic oligonucleotide
  Description of Combined DNA/RNA Molecule Synthetic oligonucleotide
gagauaagcaccucaaguatt

SEQ ID NO: 9 - DNA, 21 bp, Artificial Sequence
  Description of Artificial Sequence Synthetic oligonucleotide
  Description of Combined DNA/RNA Molecule Synthetic oligonucleotide
uacuugaggugcuuaucuctt

SEQ ID NO: 10 - DNA, 24 bp, Artificial Sequence
  Description of Artificial Sequence Synthetic oligonucleotide
  Description of Combined DNA/RNA Molecule Synthetic oligonucleotide
agaccuacggagacaauucugutt

SEQ ID NO: 11 - DNA, 25 bp, Artificial Sequence
  Description of Artificial Sequence Synthetic oligonucleotide
  Description of Combined DNA/RNA Molecule Synthetic oligonucleotide
acagaauugucuccguaggucuutt

SEQ ID NO: 12 - DNA, 21 bp, Artificial Sequence
  Description of Artificial Sequence Synthetic oligonucleotide
  Description of Combined DNA/RNA Molecule Synthetic oligonucleotide
gugaagaccuacggagacatt

SEQ ID NO: 13 - DNA, 21 bp, Artificial Sequence
  Description of Artificial Sequence Synthetic oligonucleotide
  Description of Combined DNA/RNA Molecule Synthetic oligonucleotide
ugucuccguaggucuucactt

SEQ ID NO: 14 - DNA, 21 bp, Artificial Sequence
  Description of Artificial Sequence Synthetic oligonucleotide
  Description of Combined DNA/RNA Molecule Synthetic oligonucleotide
uucugugaugugggagauctt

SEQ ID NO: 15 - DNA, 21 bp, Artificial Sequence
  Description of Artificial Sequence Synthetic oligonucleotide
  Description of Combined DNA/RNA Molecule Synthetic oligonucleotide
gaucucccacaucacagaatt

SEQ ID NO: 16 - DNA, 21 bp, Artificial Sequence
  Description of Artificial Sequence Synthetic oligonucleotide
  Description of Combined DNA/RNA Molecule Synthetic oligonucleotide
uuucccuguucucucugcatt

SEQ ID NO: 17 - DNA, 21 bp, Artificial Sequence
  Description of Artificial Sequence Synthetic oligonucleotide
  Description of Combined DNA/RNA Molecule Synthetic oligonucleotide
ugcagagagaacagggaaatt

SEQ ID NO: 18 - RNA, 21 nt, Artificial Sequence
  Description of Artificial Sequence Synthetic oligonucleotide
gguauuuaccuacaaauauuu

SEQ ID NO: 19 - RNA, 21 nt, Artificial Sequence
  Description of Artificial Sequence Synthetic oligonucleotide
auauuuguagguaaauaccuu

SEQ ID NO: 20 - DNA, 21 bp, Artificial Sequence
  Description of Artificial Sequence Synthetic oligonucleotide
  Description of Combined DNA/RNA Molecule Synthetic oligonucleotide
gauggucauuugagccacutt

SEQ ID NO: 21 - DNA, 21 bp, Artificial Sequence
  Description of Artificial Sequence Synthetic oligonucleotide
  Description of Combined DNA/RNA Molecule Synthetic oligonucleotide
aguggcucaaaugaccauctt

SEQ ID NO: 22 - DNA, 23 bp, Artificial Sequence
  Description of Artificial Sequence Synthetic oligonucleotide
  Description of Combined DNA/RNA Molecule Synthetic oligonucleotide
uggucauuugagccacuuuautt

SEQ ID NO: 23 - DNA, 23 bp, Artificial Sequence
  Description of Artificial Sequence Synthetic oligonucleotide
  Description of Combined DNA/RNA Molecule Synthetic oligonucleotide
auaaaguggcucaaaugaccatt

SEQ ID NO: 24 - DNA, 20 bp, Artificial Sequence
  Description of Artificial Sequence Synthetic oligonucleotide
  Description of Combined DNA/RNA Molecule Synthetic oligonucleotide
gugaagacuacggagacatt

SEQ ID NO: 25 - DNA, 21 bp, Artificial Sequence
  Description of Artificial Sequence Synthetic oligonucleotide
  Description of Combined DNA/RNA Molecule Synthetic oligonucleotide
ugucuccguaggucuucactt

SEQ ID NO: 26 - DNA, 21 bp, Artificial Sequence
  Description of Artificial Sequence Synthetic oligonucleotide
  Description of Combined DNA/RNA Molecule Synthetic oligonucleotide
gugaagaccuacggagacatt

SEQ ID NO: 27 - DNA, 21 bp, Artificial Sequence
  Description of Artificial Sequence Synthetic oligonucleotide
  Description of Combined DNA/RNA Molecule Synthetic oligonucleotide
ugucuccguaggucuucactt

SEQ ID NO: 28 - protein, 11 aa, Artificial Sequence
  Description of Artificial Sequence Synthetic peptide
ADMPGTVLPDK

SEQ ID NO: 29 - protein, 9 aa, Artificial Sequence
  Description of Artificial Sequence Synthetic peptide
DMPGTVLPD

SEQ ID NO: 30 - protein, 8 aa, Artificial Sequence
  Description of Artificial Sequence Synthetic peptide
DMPGTVLP

SEQ ID NO: 31 - protein, 9 aa, Artificial Sequence
  Description of Artificial Sequence Synthetic peptide
VEEGGYIAA